(19) United States
(12) Patent Application Publication
(10) Pub. No.: US 2015/0289791 A1
(43) Pub. Date: Oct. 15, 2015

(54) Pulse Oximeter Using Burst Sampling

(71) Applicant: Colin Rhodes Marcus, Cambridge, MA (US)

(72) Inventor: Colin Rhodes Marcus, Cambridge, MA (US)

(21) Appl. No.: 14/250,529

(22) Filed: Apr. 11, 2014

(51) Int. Cl. A61B 5/1455; A61B 5/00; A61B 5/0205

(52) U.S. Cl. CPC A61B 5/14551; A61B 5/0205; A61B 5/0022; A61B 5/681; A61B 5/742; A61B 5/02416

(57) ABSTRACT

This application provides a method of data collection for a pulse oximeter and an improved pulse oximeter device and improved components thereof.

### FIELD

Pulse oximeters, components thereof, and methods for their use

### BACKGROUND

Pulse oximetry is a valuable noninvasive technique for measuring several vital signs, including blood oxygen saturation (also known by the technical term peripheral capillary oxygen saturation, or SₚO₂), heart rate, and respiration rate. Pulse oximetry uses an optical measurement method that involves passing light through tissue containing blood vessels and detecting the light either transmitted through or reflected by the tissue. The pulsation of arterial blood (AC component) enables the pulse oximeter to differentiate it from the static absorption background (DC component) presented by tissue, venous blood, nonpulsating arterial blood, and the like. In general, the DC component of the signal is at least 100 times larger than the AC component.

Prior oximeters have used a significant amount of energy to run, with the best portable commercial models using more than 30 mW. As a result, pulse oximeters have faced a design choice: make the oximeter small with poor battery life or bulky with better battery life. For example, a contemporary portable oximeter capable of fitting in the pants pocket has only about 10 hours of battery life. This additionally means that hospitals typically require monitoring oximeters to be plugged in to a power source to ensure reliability, which is generally cumbersome and uncomfortable for the patient.

The basic operation of a pulse oximeter is as follows: first, a radiation source such as a light emitting diode (LED) emits visible or infrared light into a body tissue. As the light passes through the tissue, some fraction is absorbed, while the remainder arrives at a detector. A detector circuit amplifies the signal from the detector and provides the output to a calculating circuit. By comparing the absorption of light at different wavelengths, the SₚO₂ of the tissue may then be determined.

Standard oximeters use always-on detector circuits, meaning that a quiescent current or “on-current” is drawn at all times, even in the absence of any signal. Lowering the power consumption of such a circuit has the side effect of slowing it down, often reducing the bandwidth into the kilohertz range. As a result there is a minimum LED pulse duration required for the output of the detector circuit to settle enough for an accurate measurement, and pulses are spaced out evenly in time to permit the lowest bandwidth possible. Additionally, preventing the 60/120 Hz flicker from ceiling lights from aliasing into the output requires sampling at a rate above the Nyquist rate, which in this case is 240 Hz. These factors—long radiation source pulse durations due to low bandwidth, the need for fast sample rates to avoid aliasing, and a constantly active detector circuit—combine to create power hungry devices.

Given that typical LED pulse durations are on the order of 100 μs or more, feedback controlled current sources are used to maintain a constant LED brightness for the duration of each pulse. Such current sources have the downsides of taking up circuit board space, increasing oximeter cost and complexity, and losing additional power to quiescent current through the current source amplifiers. Finally, these current sources have the drawback that they suffer the same speed versus power problem as the detector circuit.

Finally, recent experimental attempts to reduce oximeter power usage have largely focused on using analog circuits to compute the oxygen saturation, instead of using a microcomputer such as a microcontroller (MCU), or digital signal processing in general. This is due to a belief that microcomputers require too much power and cannot be included in an ultra low power oximeter. The present embodiments show that this is a misconception.

### SUMMARY

In accordance with the description, one embodiment includes a pulse oximeter method comprising:
a) supplying power to at least one active component in a detector and amplifier circuit;
b) establishing a radiation state, optionally including activating a radiation source configured to emit radiation into a tissue;
c) receiving radiation that has passed through a tissue and allowing a corresponding signal to propagate through the detector and amplifier circuit;
d) taking at least one sample from the output of the detector and amplifier circuit;
e) optionally deactivating the radiation source if one was activated in part b;
f) completing a sampling burst by repeating steps b-e for all desired radiation states so that at least two radiation states are used and wherein a sampling burst has a duration of t₁;
g) interrupting power to the detector and amplifier circuit, wherein the power interruption has a duration of t₂ and wherein the detector and amplifier circuit remains on during steps a-f;
h) repeating steps a-g at least several times per second;
   wherein each cycle of steps b-e is completed in 100 μs or less and wherein t₁<t₂.

In one embodiment, the desired radiation states comprise dark.

In another embodiment, the desired radiation states comprise at least one of any of red light, dark, and infrared light, in any order.

In a further embodiment, t₁ is at least 5, 10, 100, 500, 1000, 5,000 or 10,000 times less than t₂.

In an additional embodiment, a driver circuit for an oximeter radiation source comprises:
a) a voltage source;
b) an energy storage circuit configured to receive the output of the voltage source and charge up to that voltage;
c) optionally at least one component forming a low pass network at the output of the voltage source, where the energy storage circuit may be part of said network;
d) at least one radiation source configured to emit radiation into a tissue;
e) at least one switching circuit controlling the flow of electrical current through the at least one radiation source; and
f) optionally at least one resistor in series with the radiation source.

In one embodiment, the energy storage circuitry includes at least one capacitor.

In a further embodiment, the driver circuit does not employ an amplifier between the voltage source and the radiation source.

In an additional aspect, at least one radiation source is at least one LED and wherein the driver circuit comprises at least one resistor in series with the capacitor and the radiation source.

In a further aspect of a pulse oximeter comprising a driver circuit for a radiation source and a detector and amplifier circuit,
a) the driver circuit for the radiation source comprises:
i) a voltage source;
   ii) an energy storage circuit to receive the output of the voltage source and charge up to that voltage;
   iii) optionally at least one components forming a filter network at the output of the voltage source, where the energy storage circuit may be part of said network;
   iv) at least one radiation source configured to emit radiation into a tissue;
   v) at least one switching circuit controlling the flow of electrical current through the at least one radiation source; and
   vi) optionally at least one resistor in series with the capacitor and the radiation source; and
b) a detector and amplifier circuit, configured to receive a signal from a photodetector and output a corresponding signal.

In a further mode, the detector and amplifier circuit comprises:

a) a photodetector configured to detect radiation from a tissue;

b) a first amplifier configured as a linear or nonlinear transimpedance amplifier, and configured to receive the output of the photodetector;

c) a second amplifier configured to receive the output of the first amplifier and configured to subtract a voltage reference from the signal;

d) a voltage reference generator comprising a plurality of variable voltage references, where the output of the plurality of variable voltage references is optionally connected to a multiplexing switch and the multiplexing switch is optionally configured to allow only one of the voltage references to connect to the second amplifier at a time and optionally where the output of each variable voltage reference is controlled by a microprocessor;

wherein at least one active component may be placed in a reduced power state, wherein when in the reduced power state it consumes less than or equal to 50% of the power in a full power state and

wherein the detector and amplifier circuit has a bandwidth of at least 200 kHz.

In an additional aspect, the plurality of variable voltage references correspond to variable voltage references employed for red light, infrared light, and dark.

In an additional mode, the microprocessor controls the switching circuit controlling the detector and amplifier circuit and is optionally configured to signal the switching circuit to interrupt the flow of power between sampling bursts.

In one embodiment, the microprocessor controls the multiplexing switch of the voltage reference generator.

In another embodiment, the data comprises blood oxygen saturation, heart rate, and/or respiration rate, optionally collated with metadata such as time and date data.

In a further aspect, the microprocessor performs operations during at least some of the time the detector circuit is off.

In an additional embodiment, the pulse oximeter has a display for displaying data.

In a further aspect, the data can be transmitted to a remote network, smartphone, or other computer through a physical connection or a wireless connection.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 is a flow chart providing an exemplary method of data collection for a pulse oximeter.

FIG. 2 is an exemplary embodiment of an improved pulse oximeter.

FIGS. 3A and B are exemplary embodiments of a driver circuit.

FIG. 4 shows an exemplary embodiment of a detector and amplifier circuit.

FIG. 5 shows certain burst pattern embodiments for the improved pulse oximeter.

FIG. 6 shows a physically-realized prototype pulse oximeter.

FIG. 7 shows an exemplary embodiment of the frequency response of an improved pulse oximeter.

FIG. 8 shows an exemplary waveform detected by a physically-realized improved pulse oximeter.

FIG. 9 shows an exemplary embodiment of a medical device incorporating the improved pulse oximeter.

### DESCRIPTION OF THE EMBODIMENTS

### I. Method of Data Collection for a Pulse Oximeter

The new technique of data collection will be referred to as the “burst method.” As discussed in the background, the main problem that the new method is intended to address is that of excessive power consumption by oximetry sensors. However, the burst method also has the unanticipated benefit of enabling substantial noise reduction not only in ambient light noise, but also in electrical noise internal to the oximeter circuit. This surprising result makes the burst method even more useful.

In an oximeter using the burst method, the radiation sources are pulsed in bursts, where the pulses are very close together and very short in duration. The detector circuit is fast (in some embodiments >1 MHz), and, in some embodiments, is only activated when a measurement is taking place. Moreover, by grouping the pulses tightly enough inside the burst, the aliasing problem can be solved using a process called “dark subtraction.” As a result of these factors, the burst method leads to very low power oximeters that can sample at any rate.

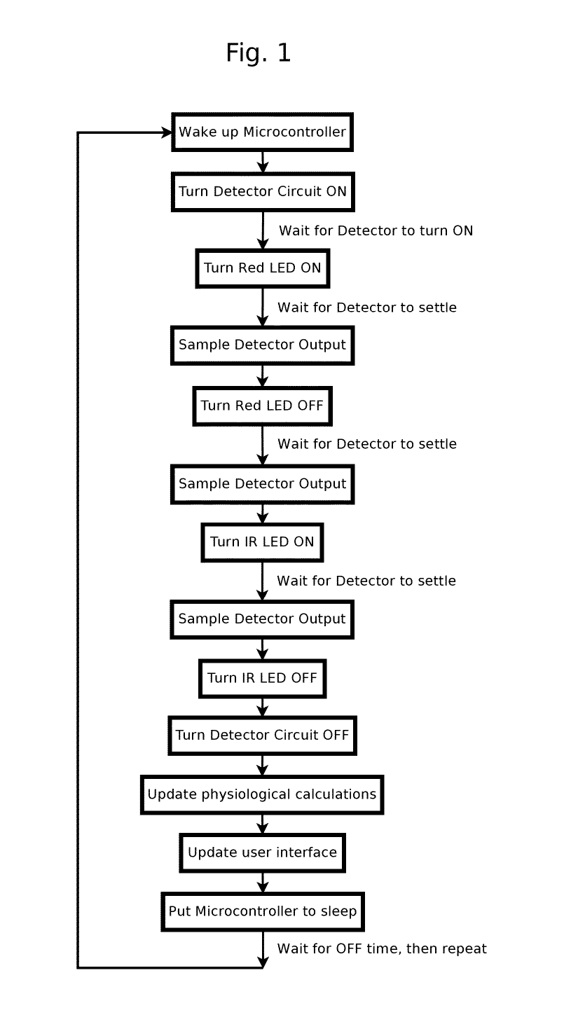

One embodiment of the burst method is shown in FIG. 1. The burst starts when a microcontroller wakes up and turns the detector circuit on. The system waits until the detector is ready, and then activates the first radiation source, which in this instance happens to be a red LED. The system waits for the output of the detector circuit to settle before sampling it with an analog to digital converter (ADC). The first radiation source is then deactivated. The detector circuit settles and the ADC takes a sample—this is the “dark pulse”, where no radiation source is active and the detector samples the ambient light. Next the second radiation source, which in this instance happens to be an infrared LED, is activated, and again the ADC waits for the detector circuit to settle, then takes a sample. No more measurements are needed, so the detector circuit is turned off. The microcontroller may use the red, dark, and infrared samples to update any physiological calculations concerning the patient, optionally updates the user interface, and finally goes to sleep. The user interface may be updated at any time in the cycle or after a certain number of cycles had occurred (for example, after 5, 10, 15, 20, or 25 cycles). After some “Off Time”, the microcontroller wakes up and the cycle repeats.

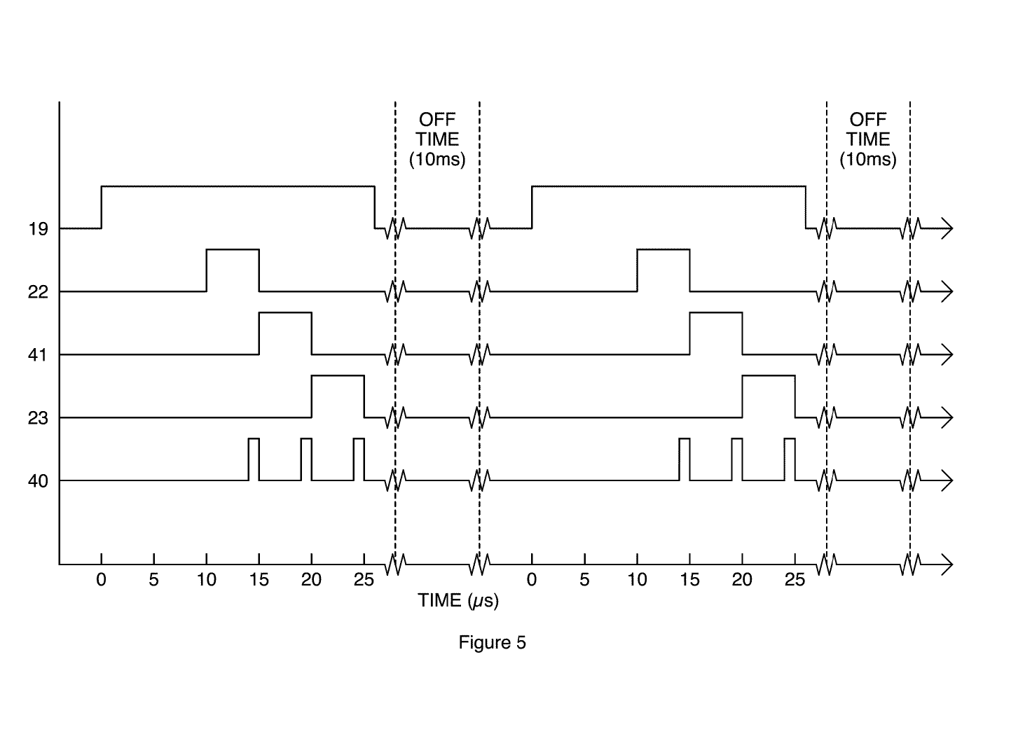

FIG. 5 shows an exemplary burst method pattern. Here, the horizontal axis represents time, while the ON/OFF states of the components are shown in the vertical. Signal 19 controls the state of the detector circuit. Signals 22, 41, and 23 each control a radiation state. To continue the discussion of the particular embodiment presented in FIG. 1, signal 22 represents, in this instance, the red LED, while 41 represents, in this instance, the dark pulse, and 23 represents, in this instance, the infrared LED; however, the ordering of pulses may be rearranged. Signal 40 shows spikes to indicate when the ADC is sampling the detector circuit.

In this embodiment, at time t=0, the detector circuit is activated. At t=10 μs, the detector circuit is ready and the red LED is turned on. At t=14 μs the detector circuit has settled and the ADC takes a sample. At t=15 μs the red LED turns off and the dark pulse begins. At t=19 μs the detector circuit has settled and the ADC takes a sample. At t=20 μs the infrared LED is turned on. At t=24 μs the detector circuit has settled and the ADC takes a sample. At t=25 μs, the infrared LED and the detector circuit are turned off, and the burst ends. The system sleeps for the Off Time, which here is 10 ms, corresponding to a burst frequency of 100 Hz. As shown, the cycle repeats after the Off Time has expired.

The average power expenditure of this burst pattern can be estimated. For example, assume that the detector circuit uses 20 mA when active, and each LED consumes 50 mA when active. Considering that the detector is active for 25 μs, each LED is active for 5 μs, and the repetition rate is 100 Hz (from the Off Time), one finds that this oximeter uses 100 μA of current, or 300 μW for a typical 3V power supply. This is about 100 times less power than the best commercial oximeters, and 10 times less than the best experimental oximeter known to the inventor.

In the burst method, the dark pulse may be sampled directly by the ADC in the same manner as signals with an active radiation source. By subtracting the dark signal out of the illuminated signals, low frequency ambient light interference can be removed (dark subtraction). However, the situation is actually more complex than it first appears: simple dark subtraction does not actually reduce noise, instead it alters the spectral distribution according to Equation 1:

ΔS=2*δ̂2*Sin [π*τ*f]̂2 Equation 1

where

δ=noise magnitude

τ=sample separation in time

f=frequency

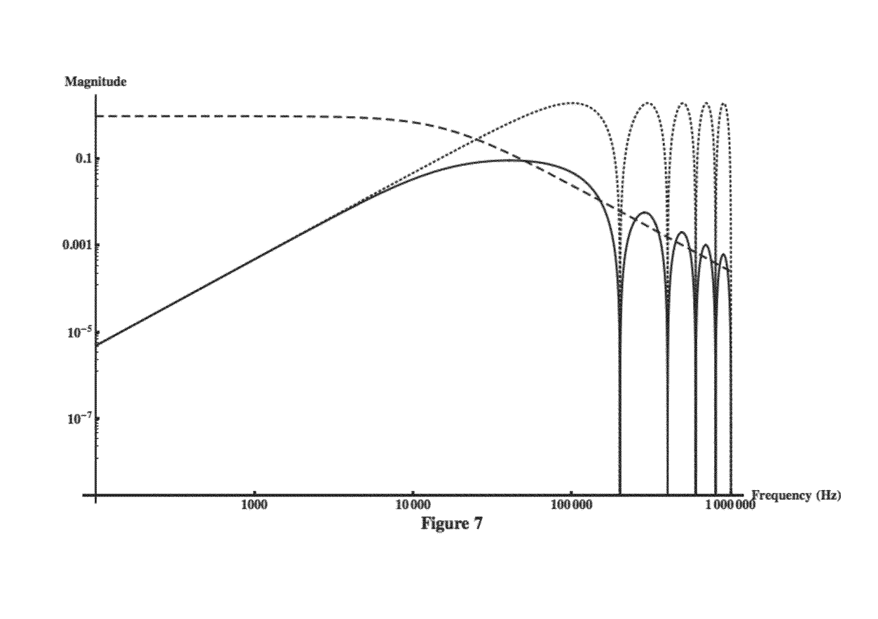

FIG. 7 provides a log-log plot of the frequency response of dark subtraction with τ=5 μs (dotted line), a low pass filter with corner frequency f_{c}=16 kHz (dashed line), and the combination of the two (solid line). Dark subtraction alters a noise signal as if it were being passed through an energy conserving filter with an oscillatory frequency response. At some frequencies the noise density is attenuated, while at other frequencies the noise density actually increases. More precisely, the frequency response has maxima at f=n/(2τ) where n is odd, and minima where n is even. Since one of the minima is at zero frequency, dark subtraction can completely remove DC signals.

For frequencies between zero and 1/(4τ), dark subtraction behaves like a first order high pass filter with a rolloff of 20 dB/decade. In order to reduce overall noise, an additional filter such as a low pass filter can be added such that frequencies above 1/(4τ) are also attenuated. The end result is that the noise experiences both the high pass and low pass filters, while the signal experiences only the low pass filter. This effect extends not only to the compensation of ambient light interference, but also to the electrical noise occurring inside the detector circuit itself. By lowering τ to the smallest value possible, the burst method exploits this effect while simultaneously achieving an extremely small duty cycle, and thus minimal power consumption.

In order for electrical noise to be attenuated by dark subtraction, a condition may be fulfilled. The noise in each pulse may be “common mode,” meaning that the noise sources may be the same. For example, imagine that two potentiometers are placed in parallel between a noisy voltage rail and ground, and the voltage at each center tap is measured. Noise on the voltage rail will show up equally across each potentiometer (common mode), and thus subtracting one voltage from the other will remove the noise. By comparison, the thermal noise in each potentiometer is uncorrelated, and subtraction is not effective (not common mode).

The ideal burst method detector circuit tries to use the same signal paths between pulses to the greatest possible extent, while also optimizing for speed in order to cut power consumption.

### II. Improved Pulse Oximeter

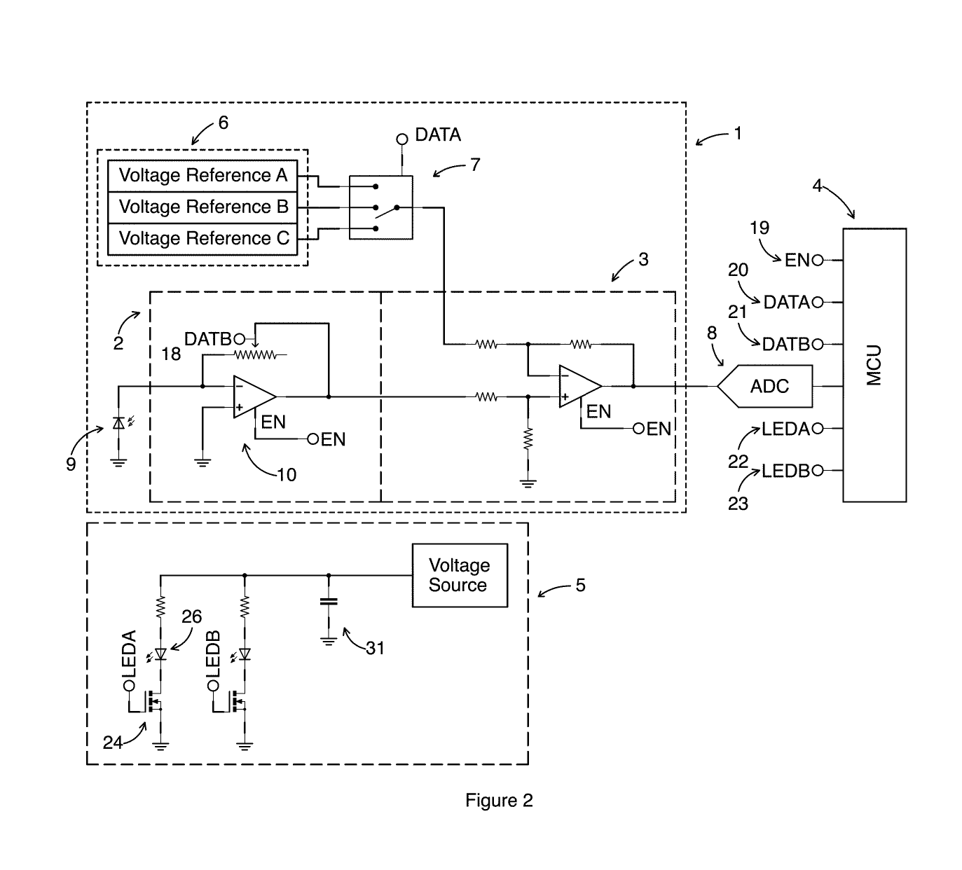

One embodiment of an improved pulse oximeter configured for using the burst method is provided in FIG. 2. The system as a whole is comprised of the following subsystems: a driver circuit 5 configured to emit light into a tissue, a detector and amplifier circuit 1 configured to detect said light and convert it into a signal suitable for analog-to-digital conversion, an ADC 8 configured to produce a digital code representative of the signal received from 1, and a digital processor 4 configured to receive the digital code and operate on it to produce data representing an oxygen saturation.

In one embodiment, the pulse oximeter comprises at least one active component may be placed in a reduced power state, wherein when in the reduced power state it consumes less than or equal to 50% of the power in a full power state. In another embodiment, the pulse oximeter comprises at least one active component that may be placed in a reduced power state, wherein when in the reduced power state it consumes less than or equal to 45%, 40%, 35%, 30%, 25%, 20%, 15%, 105, or 5% of the power in a full power state. In another embodiment, the pulse oximeter comprises at least one active component that may be placed in an off state where it consumes no power while in that state.

In one embodiment, the active component that may be placed in a reduced power state and/or an off state may be chosen from operational amplifiers, amplifiers, digital potentiometers, voltage regulators, voltage references, DACs, ADCs, microcontrollers, processors, or any other component that requires an external source of power in order to operate.

A. Driver Circuit

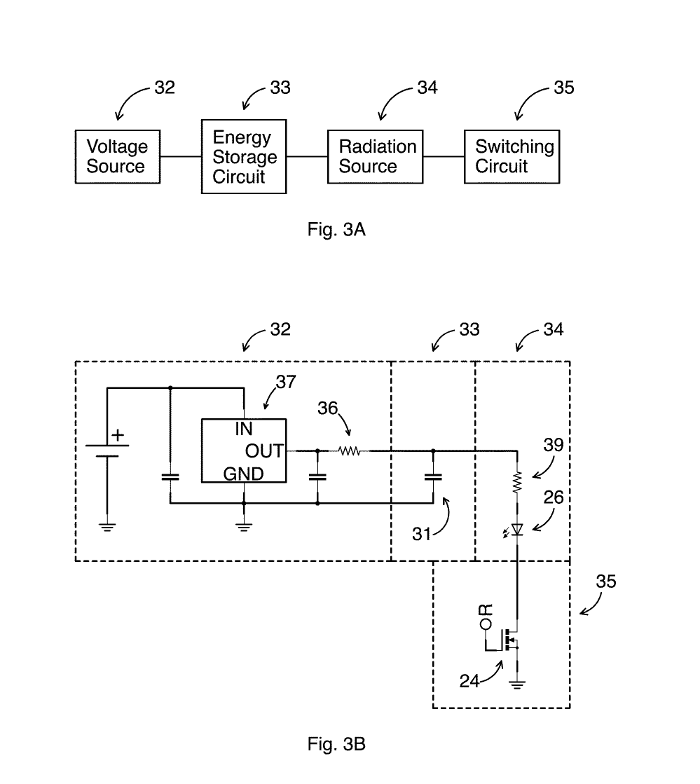

The driver circuit can be broken down into 4 sections as shown in FIG. 3A: a voltage source, energy storage circuit, radiation source, and a switching circuit, which may be accompanied by other optional elements. The voltage source 32 provides a stable voltage output which is used to charge an energy storage circuit 33. The energy storage circuit 33 acts as an energy reservoir capable of powering the radiation source 34. Switching circuit 35 is in series with 34 and controls the flow of current through 34.

In one embodiment, the driver circuit is constructed according to FIG. 3B. The operation of the driver using the burst method is as follows. The switches 35 are opened, preventing current from flowing through the radiation sources 34. Linear voltage regulator 37 charges energy storage capacitor 31 through resistor 36. When a pulse of red light is desired, a high logic state is set on signal R, which controls the gate of MOSFET switch 24. This causes the MOSFET switch 24 to conduct, allowing current to flow from 31 and through LED 26, causing it to emit light. When the desired data has been collected, LED 26 is turned off again by setting R low. This procedure may be repeated for any number of LEDs and switches. FIG. 2 includes an example of a driver with two LEDs and switches.

In one embodiment, LED pulses are very fast and presented in a burst mode, with individual pulses in some embodiments on the order of 5-10 μs. This allows for an improved driver circuit strategy. In one embodiment, a voltage source may be used to charge an energy storage circuit, which energizes at least one LED in short bursts by means of switches. By selecting a suitably large capacitance for the energy storage circuit, the change in current through an LED during an activation can be reduced to arbitrarily low levels. This relationship is estimated in Equation 2:

C≧(I*Δt)/(V*ΔV) Equation 2:

where

C=capacitance

I=radiation source current,

Δt=sampling window jitter,

V=capacitor voltage,

ΔV=percentage voltage change during the pulse

For example, an LED is activated at 50 mA current, powered by an energy storage capacitor charged to 3V. The analog to digital converter has a sampling window jitter of 100 ns and a resolution of 16 bits, equating to a ΔV of 1/(2̂12). The energy storage capacitor may then be greater than 110 μF to keep current variations below 1 least significant bit.

This simpler driver method has substantial advantages over prior art drivers. First, it has very fast turn-on and settling times compared to feedback based drivers. Second, in one embodiment, the only quiescent current losses are in the voltage source, and these can be minimized if the voltage source is configured to more slowly charge the energy storage circuit through a resistance. Slow charging both enables the use of more efficient low-bandwidth regulator circuits for the voltage source and prolongs battery life if that is the system power source. Third, this driver can generally be generally be made smaller and less expensively compared to prior art drivers.

1. Voltage Source

The voltage source is defined as a circuit capable of outputting a stable voltage. In one embodiment, the voltage source may be chosen from linear voltage regulators, digital-to-analog converters, buffered potentiometers, and the like. In another embodiment, components with less stability may be used, such as batteries; in some embodiments care is taken to ensure any instability is compensated or otherwise accounted for.

In one embodiment, the voltage source is connected to additional components such as low pass filters to remove noise from the output. In another embodiment, an impedance such as resistor 36 suitable for forming a low pass filter with the energy storage capacitor 31 may be added to the output of the voltage source. Furthermore, in one embodiment, any active components in the voltage source may have the ability to be disabled or otherwise placed in a low power state. Active component refers to any component that draws power just by being turned on or “active”. For instance, this may, in some embodiments, include amplifiers, op amps, microcontrollers, anything that requires a power source to operate, and thus can be turned off by interrupting the power.

2. Energy Storage Circuitry

The driver additionally includes energy storage circuitry configured to receive the output of the voltage source and charge up to that voltage. In one embodiment, the energy storage circuitry may include at least one capacitor configured to receive the output of the voltage source. This energy storage circuitry acts as energy storage to power the fast LED pulses used by the burst mode method. In one embodiment, the voltage source is configured to have a substantial output impedance such as resistor 36, configured to form a low pass filter with the energy storage capacitor 31. This serves to attenuate noise and isolate the capacitor from any high frequency fluctuations coming from the voltage source.

3. At Least One Radiation Source Configured to Emit Radiation into a Tissue

The radiation source may be chosen from at least one light emitting diode (“LED”) 26 configured to emit light into a tissue. The at least one LED is configured to use the energy storage capacitor as a power source, and may have an additional resistive impedance 39 placed in series for current limiting purposes.

In one embodiment, a single LED may have an infrared light mode and a red light mode. In another embodiment, two LEDs are used, a first with an infrared mode and a second with a red light mode. It is understood that red and infrared are specifically mentioned because they are the most typical wavelengths used in oximetry; however other wavelengths can be substituted if desired.

4. At Least One Switching Circuit with at Least One Switch Controlling the Flow of Electrical Current Through the at Least One Radiation Source

The switching circuit comprises at least one switch configured such that individual LEDs or sets of LEDs can be turned on or off. In one embodiment, these switches may be embodied by transistors such as bipolar junction transistors (“BJTs”). In another embodiment, the switches may be metal oxide semiconductor field effect transistors (“MOSFETs”), offering a benefit due to their reduced gate drive requirements. An embodiment of this MOSFET switching circuit is shown in FIG. 3B, where MOSFET 24 is controlled by gate signal “R”.

B. Detector and Amplifier Circuit

The detector and amplifier (“D&A”) circuit is comprised of a photodetector which senses incoming light, and then one or more amplifiers that condition the signal prior to its conversion into digital format by an ADC.

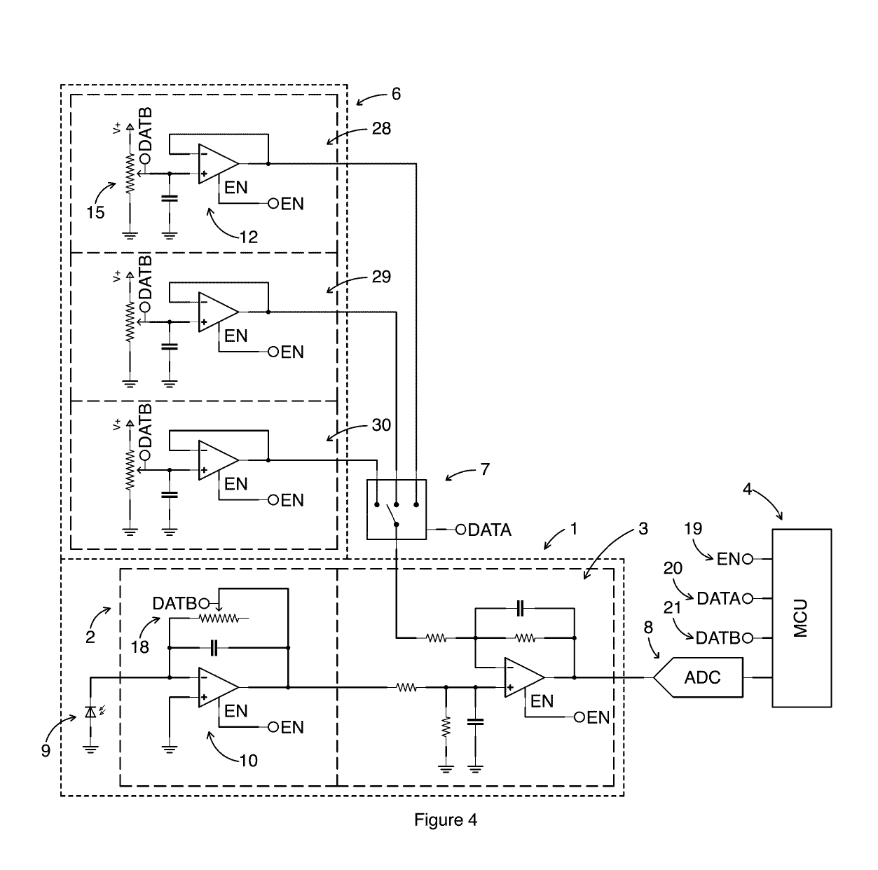

In one embodiment, the oximeter is operated according to the burst method. In one embodiment, since the LED radiation sources are activated only for brief intervals, the D&A circuit may be designed such that the signal presented to ADC 8 settles within the LED activation time. FIG. 4 presents an example of such a D&A circuit.

The term “settling time” is here defined to indicate the time needed for a signal to approach its steady state, such that the signal presented to the ADC is within 1 least significant bit of its final value.

In order to take full advantage of the burst method, in one embodiment, active components in the D&A circuit have the ability to be placed in a low power state. In one embodiment, this is done using an enable pin on the operational amplifiers (“op amps”), marked “EN” and connected to digital EN signal 19. When the EN signal is high the op amps are turned on, when EN is low the op amps are turned off.

In FIG. 4, light incident on photodiode 9 produces a corresponding current. Transimpedance amplifier 2 receives this current and converts it to a corresponding voltage. Differencing amplifier 3 receives this voltage and subtracts a reference voltage from it, then amplifies it. ADC 8 receives this amplified voltage, converts it into a digital code, and provides it to processor 4 for analysis.

1. Photodetector

The photodetector may be any electrical component that produces a change in response to incoming light. In one embodiment, photodiodes are used for their fast response times. Photodiode 9 in FIG. 4 is configured to produce a positive voltage at the output of transimpedance amplifier 2.

2. Transimpedance Amplifier

The transimpedance amplifier is configured to receive the current signal from photodiode 9 and convert it into a corresponding voltage according to Equation 3:

V=−IR Equation 3

where

V=output voltage

I=current through photodiode 9

R=resistance of potentiometer 18

In one embodiment, potentiometer 18 is configured to be adjustable according to a digital signal DATB or signal 21 transmitted by processor 4. Thus, processor 4 can adjust the gain of this circuit, in one embodiment in order to maximize gain while avoiding saturation of op amp 10.

It is also possible to build a nonlinear transimpedance amplifier, for example by replacing potentiometer 18 with a diode. This has potential benefits in both reducing the amount of math performed by processor 4 and increasing the dynamic range of the transimpedance amplifier 2, but also has the drawback that the gain is not controllable.

3. Differencing Amplifier

Differencing amplifier 3 is configured to receive an input voltage from 2, subtract a reference voltage from it, apply a gain to it, and present the resulting voltage at the output. This process is provided by Equation 4:

V=G(Vᵢₙ−V_{ref}) Equation 4

where

V=output voltage

G=gain

Vᵢₙ=input voltage

V_{ref}=reference voltage

The purpose of subtracting a reference voltage from the signal is to increase the sensitivity of the device. The signal consists of a large DC signal added to the AC signal of interest. By removing the DC signal, the remaining AC signal can be amplified more than would otherwise be possible. Moreover, the processor knows the magnitude of the subtracted signal and can add it back on to the signal seen by the ADC.

4. Voltage Reference Generator

The voltage reference generator (VRG) provides the reference voltage used by the differencing amplifier. In one embodiment, there is one VRG, which changes between different reference voltages depending on which radiation state is being used (i.e. red, IR, or dark). In the embodiment of FIG. 4, there is a separate physical VRG for each radiation state, VRGs 28, 29, and 30, collectively referred to as VRG module 6, and a multiplexing switch 7 selects one at a time to connect to the differencing amplifier. This selection is made according to a control signal from the processor, here denoted as DATA, or signal 20.

VRG 28 consists of a digital potentiometer 15 buffered by a unity gain amplifier 12. The voltage is controlled by processor 4, which sets the digital potentiometer via DATB, or signal 21. After changing the potentiometer voltage setting, the system may wait for it to settle before attempting a measurement. The multiple VRGs with a multiplexing switch allows for the burst method of taking a tight cluster of measurements.

The embodiment of FIG. 4 provides a lower noise solution when used in conjunction with the burst method. Since the burst method provides the equivalent of high pass filtering on common mode signals, a low cost linear voltage regulator can be used as the positive supply rail for the potentiometers. The noninverting input to each op amp is low pass filtered, and the supply rail is common mode, meaning that supply rail noise can be reduced by over an order of magnitude. The ability to use this kind of design is a major benefit of the burst method.

5. Analog to Digital Converter

The analog to digital converter should be capable of taking samples quickly enough to support the burst method, and at sufficient resolution to produce useful data. In general, the resolution should be fine enough that the AC part of the oximetry signal is spread across at least several levels of the ADC step response.

In one embodiment, preceding stages of the amplifier deliberately introduce a substantial amount of noise into the signal in a technique known as dithering. This introduction of randomness causes the signal to jump between ADC levels, and if enough samples are taken, the value of the signal can be determined more precisely than if there was too little noise and the signal was trapped at one level of the ADC.

In another embodiment, the ADC includes a data buffer that stores recently measured values. This allows the ADC to take several samples in succession before sending the data to the processor.

Reference will now be made in detail to the present exemplary embodiments, examples of which are illustrated in the accompanying drawings. Wherever possible, the same reference numbers will be used throughout the drawings to refer to the same or like parts. Other embodiments will be apparent to those skilled in the art from consideration of the specification and practice disclosed herein. The embodiments are further explained in the following examples. These examples do not limit the scope of the claims, but merely serve to clarify certain embodiments. It is intended that the specification and examples be considered as exemplary only, with a true scope and spirit being indicated by the following claims.

### EXAMPLES

### Example 1

### A Working Pulse Oximeter

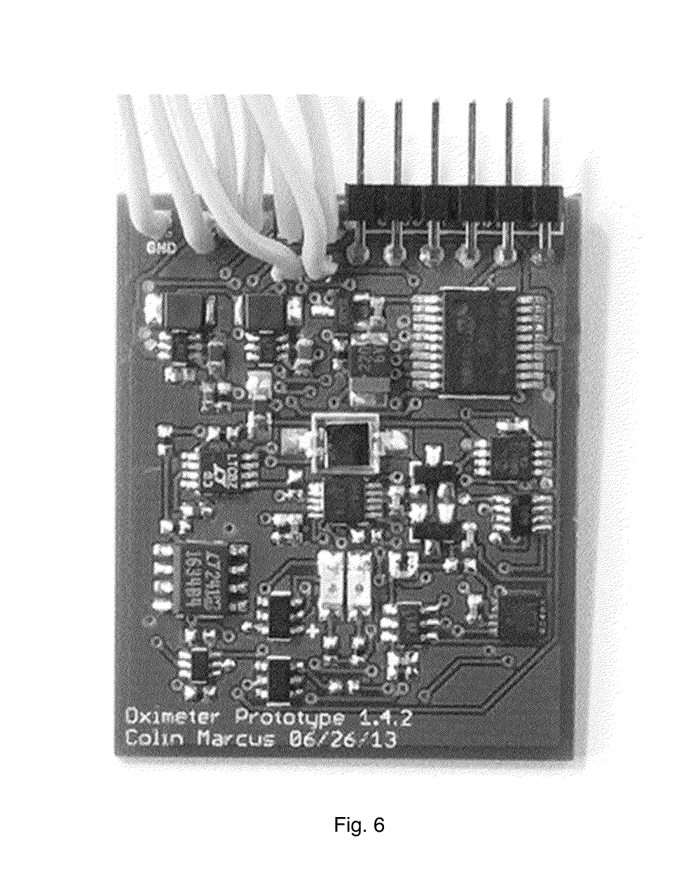

A prototype oximeter has been constructed to test the working principles of the burst method, and a picture is provided in FIG. 6. This prototype was designed for reflectance mode oximetry, meaning that the radiation source and the detector are on the same side of the tissue being measured. It should be noted that this device was a proof of concept, and did not contain the computer algorithm to convert the raw data into a numerical oxygen saturation measurement, but rather was used to show that a device designed for use with the burst method could provide acceptable detection.

This oximeter used a variation of the driver circuit described previously, where in this case the voltage source was a DC-DC voltage booster feeding into a low-pass filter to attenuate high frequency energy. The radiation source consisted of a 660 nm LED and a 940 nm LED, each in series with a resistor setting its current to 50 mA. The switches controlling the LEDs were N channel MOSFETs.

The detector circuit employed a logarithmic transimpedance amplifier as mentioned in section II.B.2 above. This method was used because it was unknown how strong of a signal would be detected, and thus the greater dynamic range of a log amp was desirable. The differencing amplifier used one VRG, and for this reason was slower than other embodiments of the burst method. Both amplifier circuits used active components with a shutdown capability. Because of the single VRG, the sample separation time τ was 30 μs, although the pulse duration was only 10 μs. The final power usage of the circuit was measured at about 1.5 mW.

### Example 2

### Testing of a Working Pulse Oximeter

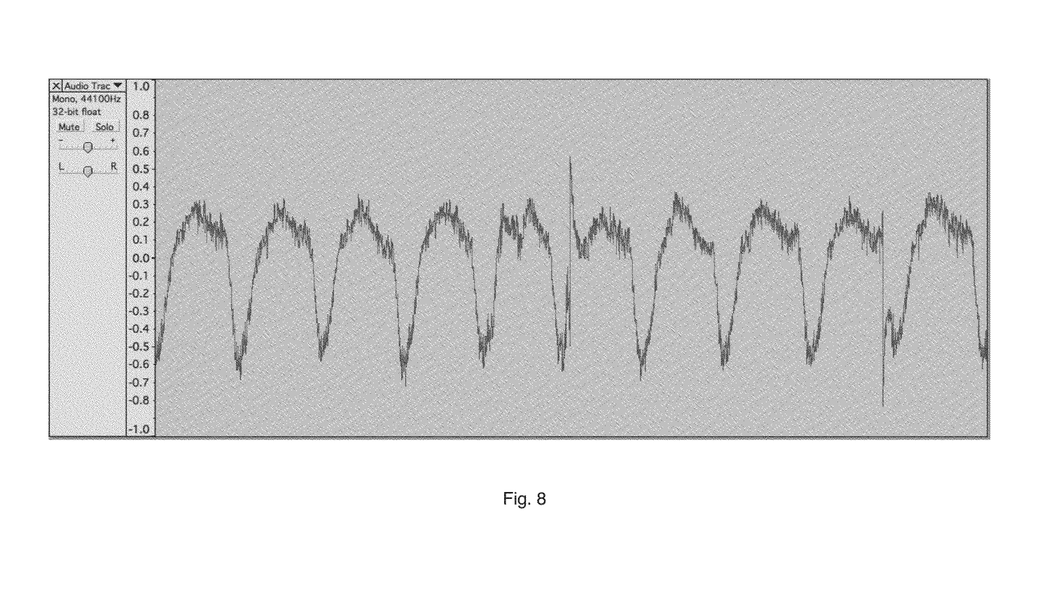

The oximeter was configured to output data in real time by sending the measured signal level to a digital to analog converter (DAC). Converting the output to an analog signal facilitated viewing the signal on an oscilloscope or recording it using a standard microphone input port. FIG. 8 shows an example waveform recorded by hooking the prototype up to a laptop and using audio recording software. The system successfully recorded the subject's heartbeat as about 1.15 Hz, or 70 beats/minute. The waveform also shows two sharp vertical glitches—these are a result of the VRG changing its output level to adapt to changing light levels.

This prototype oximeter, having succeeded in collecting raw data of sufficient quality, was not used to calculate the oxygen saturation. However, it could have done so by implementing a simple algorithm. The algorithm is given in Equation 5:

$${SO}_{2} ( {R}_{os} ) = \frac{( {ɛ}_{r , Hb} - {ɛ}_{ir , Hb} {R}_{os} )}{( {ɛ}_{r , Hb} - {ɛ}_{r , HbO 2} + ( {ɛ}_{ir , HbO 2} - {ɛ}_{ir , Hb} ) {R}_{os} )} \qquad Equation 5$$

where ε represents the molar extinction coefficients of hemoglobin and oxyhemoglobin in red (660 nm) and infrared (940 nm) light. Rₒₛ is calculated from the raw data using Equation 6:

Rₒₛ=(i_{AC,R}/I_{DC,R})/(i_{AC,IR}/I_{DC,IR}) Equation 6:
where i_{AC} represents the AC component of the signal and I_{DC} the DC component, each in both red and infrared light.

### Example 3

### A Watch-Band Mounted Pulse Oximeter

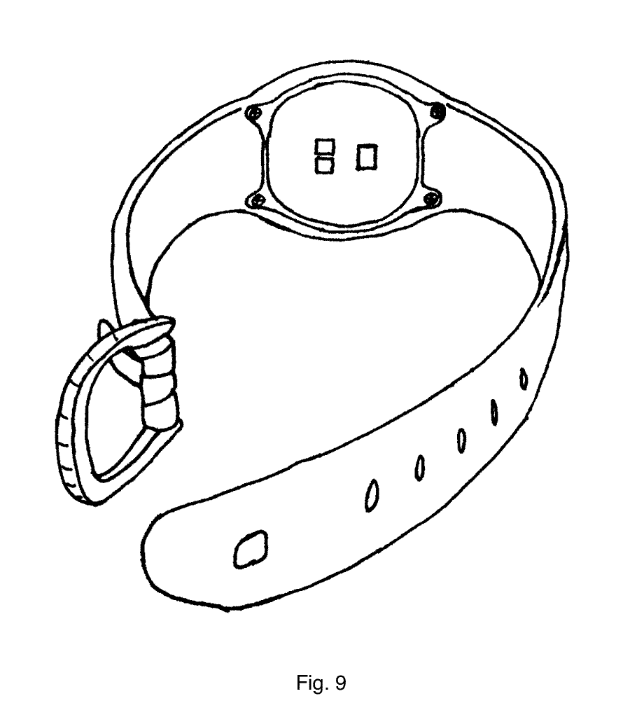

One embodiment of a device incorporating an oximeter using the burst method is provided in FIG. 9. In this embodiment, the oximeter is designed to be worn in the same manner as a wristwatch, and operates in the reflectance mode. By using the burst method and suitably optimized hardware, the average power usage is expected to be reduced to below 1 mW—low enough to operate continuously for 30 days on a rechargeable 230 mAh coin cell. The oximeter further contains an integrated radio, allowing it to transmit data wirelessly to a hospital network, smartphone, or other computer.

Such an oximeter has multiple applications. In the hospital, it allows for continual monitoring of patients vitals—reducing the number of missed significant medical events while simultaneously reducing the workload on hospital staff. Normally patients have to be woken up repeatedly throughout the night for their vitals to be taken, but with this oximeter they can sleep uninterrupted, reducing unnecessary stress. Moreover, the wireless nature of the device allows for increased mobility since the patient is not attached to anything.

Outside the hospital, one predicted use is in the monitoring and detection of intermittent heart conditions which may not be present when the patient is actually in the doctor's office. By wearing the oximeter and having it send the data to a smartphone, it becomes possible to collect medical data for a long period of time during everyday life. Moreover, if a life-threatening event is detected, the smartphone can be programmed to alert the patient and/or doctor or other emergency services even in the event that the patient has already become incapacitated.

### Example 4

### Certain Exemplary Embodiments

This example provides a variety of items serving as additional exemplary embodiments.

Item 1. A pulse oximeter method comprising:
a) supplying power to at least one active component in a detector and amplifier circuit;
b) establishing a radiation state, optionally including activating a radiation source configured to emit radiation into a tissue;
c) receiving radiation that has passed through a tissue and allowing a corresponding signal to propagate through the detector and amplifier circuit;
d) taking at least one sample from the output of the detector and amplifier circuit;
e) optionally deactivating the radiation source if one was activated in part b;
f) completing a sampling burst by repeating steps b-e for all desired radiation states so that at least two radiation states are used and wherein a sampling burst has a duration of t₁;
g) interrupting power to the detector and amplifier circuit, wherein the power interruption has a duration of t₂ and wherein the detector and amplifier circuit remains on during steps a-f;
h) repeating steps a-g at least several times per second;
   wherein each cycle of steps b-e is completed in 100 μs or less and wherein t₁<t₂.

Item 2. The method of item 1, wherein the desired radiation states comprise red light.

Item 3. The method of any one of items 1-2, wherein the desired radiation states comprise infrared light.

Item 4. The method of any one of items 1-3, wherein the desired radiation states comprise dark.

Item 5. The method of item 1, wherein the desired radiation states are red light and infrared light, in any order.

Item 6. The method of item 1, wherein the desired radiation states comprise at least one of any of red light, dark, and infrared light, in any order.

Item 7. The method of any one of items 1-6, wherein t₁ is at least 10, 100, 500, 1000, 5,000 or 10,000 times less than t₂.

Item 8. The method of any one of items 1-7, wherein at least one occurrence of step d comprises taking at least two samples from the output of the detector and amplifier circuit.

Item 9. The method of any one of items 1-8, wherein the radiation source is at least one LED light.

Item 10. The method of any one of items 1-9, wherein the radiation source comprises two LED lights, a first capable of emitting red light and a second capable of emitting infrared light.

Item 11. The method of any one of items 1-10, wherein the detector and amplifier circuit comprises a photodetector.

Item 12. A driver circuit for an oximeter radiation source comprising
a) a voltage source;
b) an energy storage circuit configured to receive the output of the voltage source and charge up to that voltage;
c) optionally at least one component forming a low pass network at the output of the voltage source, where the energy storage circuit may be part of said network;
d) at least one radiation source configured to emit radiation into a tissue;
e) at least one switching circuit controlling the flow of electrical current through the at least one radiation source; and
f) optionally at least one resistor in series with the radiation source.

Item 13. The driver circuit of item 12, wherein the energy storage circuitry includes at least one capacitor.

Item 14. The driver circuit of any one of items 12-13, wherein the driver circuit does not employ an amplifier between the voltage source and the radiation source.

Item 15. The driver circuit of any one of items 12-14, wherein the voltage source is fixed.

Item 16. The driver circuit of any one of items 12-15, wherein the voltage source is variable.

Item 17. The driver circuit of any one of items 12-16, wherein the voltage source is a regulator or a digital-to-analog converter.

Item 18. The driver circuit of any one of items 12-17, wherein the voltage source is a lithium ion battery.

Item 19. The driver circuit of item 18, wherein the voltage source is a rechargeable lithium ion battery.

Item 20. The driver circuit of any one of items 12-19, wherein the at least one radiation source is at least one LED and wherein the driver circuit comprises at least one resistor in series with the capacitor and the radiation source.

Item 21. A pulse oximeter comprising a driver circuit for a radiation source and a detector and amplifier circuit, wherein
a) the driver circuit for the radiation source comprises:
   i) a voltage source;
   ii) optionally an energy storage circuit to receive the output of the voltage source and charge up to that voltage;
   iii) optionally components forming a low pass network at the output of the voltage source, where the energy storage circuit may be part of said network;
   iv) at least one radiation source configured to emit radiation into a tissue;
   v) at least one switching circuit controlling the flow of electrical current through the at least one radiation source; and
   vi) optionally at least one resistor in series with the capacitor and the radiation source; and
b) a detector and amplifier circuit, configured to receive a signal from a photodetector and output a corresponding signal.

Item 22. The pulse oximeter of item 21, wherein the detector and amplifier circuit comprises:
a) a photodetector configured to detect radiation from a tissue;
b) a first amplifier configured as a linear or nonlinear transimpedance amplifier, and configured to receive the output of the photodetector;
c) a second amplifier configured to receive the output of the first amplifier and configured to subtract a voltage reference from the signal;
d) a voltage reference generator comprising a plurality of variable voltage references, where the output of the plurality of variable voltage references is optionally connected to a multiplexing switch and the multiplexing switch is optionally configured to allow only one of the voltage references to connect to the second amplifier at a time and optionally where the output of each variable voltage reference is controlled by a microprocessor;
wherein at least one active component may be placed in a reduced power state, wherein when in the reduced power state it consumes less than or equal to 50% of the power in a full power state and

wherein the detector and amplifier circuit has a bandwidth of at least 200 kHz.

Item 23. The detector and amplifier circuit of item 22, wherein the plurality of variable voltage references correspond to variable voltage references employed for red light and infrared light.

Item 24. The detector and amplifier circuit of item 22, wherein the plurality of variable voltage references correspond to variable voltage references employed for red light, infrared light, and dark.

Item 25. The detector and amplifier circuit of any one of items 22-24, wherein the photodetector is capable of operating in a reflectance mode.

Item 26. The detector and amplifier circuit of item 22-25, wherein the photodetector is capable of operating in a transmittance mode.

Item 27. The detector and amplifier circuit of any one of items 22-26, wherein at least one switching circuit controls the detector and amplifier circuit.

Item 28. The pulse oximeter of item 22, further comprising an analog-to-digital converter configured to receive the signal from the output of the detector and amplifier circuit.

Item 29. The pulse oximeter of item 28, further comprising a microprocessor that is capable of performing operations on the digital signal from the analog-to-digital converter and produce data.

Item 30. The pulse oximeter of any one of items 28-29, wherein the microprocessor controls the switching circuit controlling the detector and amplifier circuit and is optionally configured to signal the switching circuit to interrupt the flow of power between sampling bursts.

Item 31. The pulse oximeter of items 28-30, wherein the microprocessor controls the multiplexing switch of the voltage reference generator.

Item 32. The pulse oximeter of any one of items 28-31, wherein the data comprises blood oxygen saturation, heart rate, and/or respiration rate, optionally collated with metadata such as time and date data.

Item 33. The pulse oximeter of any one of items 28-32, wherein the microprocessor performs operations during at least some of the time the detector circuit is off

Item 35. The pulse oximeter of any one of items 28-33, wherein the pulse oximeter has a display for displaying data.

Item 36. The pulse oximeter of any one of items 28-35, wherein the data can be transmitted to a remote network, smartphone, or other computer through a physical connection or a wireless connection.

Item 37. The pulse oximeter of item 36, wherein the physical connection is a temporary connection.

Item 38. The pulse oximeter of any one of items 21-37, wherein the pulse oximeter comprises at least one active component that may be placed in a reduced power state, wherein when in the reduced power state it consumes less than or equal to 45%, 40%, 35%, 30%, 25%, 20%, 15%, 105, or 5% of the power in a full power state.

Item 39. The pulse oximeter of any one of items 31-38, wherein the pulse oximeter comprises at least one active component that may be placed in an off state where it consumes no power while in that state.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiments may be practiced in many ways and the claims include any equivalents thereof.

1. A pulse oximeter method comprising:
a) supplying power to at least one active component in a detector and amplifier circuit;
b) establishing a radiation state, optionally including activating a radiation source configured to emit radiation into a tissue;
c) receiving radiation that has passed through a tissue and allowing a corresponding signal to propagate through the detector and amplifier circuit;
d) taking at least one sample from the output of the detector and amplifier circuit;
e) optionally deactivating the radiation source if one was activated in part b;
f) completing a sampling burst by repeating steps b-e for all desired radiation states so that at least two radiation states are used and wherein a sampling burst has a duration of t₁;
g) interrupting power to the detector and amplifier circuit, wherein the power interruption has a duration of t₂ and wherein the detector and amplifier circuit remains on during steps a-f;
h) repeating steps a-g at least several times per second;
wherein each cycle of steps b-e is completed in 100 μs or less and wherein t₁<t₂.

2. The method of claim 1, wherein the desired radiation states comprise dark.

3. The method of claim 1, wherein the desired radiation states comprise at least one of any of red light, dark, and infrared light, in any order.

4. The method of claim 2, wherein t₁ is at least 5, 10, 100, 500, 1000, 5,000 or 10,000 times less than t₂.

5. A driver circuit for an oximeter radiation source comprising
a) a voltage source;
b) an energy storage circuit configured to receive the output of the voltage source and charge up to that voltage;
c) optionally at least one component forming a low pass network at the output of the voltage source, where the energy storage circuit may be part of said network;
d) at least one radiation source configured to emit radiation into a tissue;
e) at least one switching circuit controlling the flow of electrical current through the at least one radiation source; and
f) optionally at least one resistor in series with the radiation source.

6. The driver circuit of claim 5, wherein the energy storage circuitry includes at least one capacitor.

7. The driver circuit of claim 5, wherein the driver circuit does not employ an amplifier between the voltage source and the radiation source.

8. The driver circuit of claim 5, wherein the at least one radiation source is at least one LED and wherein the driver circuit comprises at least one resistor in series with the capacitor and the radiation source.

9. A pulse oximeter comprising a driver circuit for a radiation source and a detector and amplifier circuit, wherein
a) the driver circuit for the radiation source comprises:
i) a voltage source;
ii) an energy storage circuit to receive the output of the voltage source and charge up to that voltage;
iii) optionally at least one components forming a filter network at the output of the voltage source, where the energy storage circuit may be part of said network;
iv) at least one radiation source configured to emit radiation into a tissue;
v) at least one switching circuit controlling the flow of electrical current through the at least one radiation source; and
vi) optionally at least one resistor in series with the capacitor and the radiation source; and
b) a detector and amplifier circuit, configured to receive a signal from a photodetector and output a corresponding signal.

10. The pulse oximeter of claim 9, wherein the detector and amplifier circuit comprises:
a) a photodetector configured to detect radiation from a tissue;
b) a first amplifier configured as a linear or nonlinear transimpedance amplifier, and configured to receive the output of the photodetector;
c) a second amplifier configured to receive the output of the first amplifier and configured to subtract a voltage reference from the signal;
d) a voltage reference generator comprising a plurality of variable voltage references, where the output of the plurality of variable voltage references is optionally connected to a multiplexing switch and the multiplexing switch is optionally configured to allow only one of the voltage references to connect to the second amplifier at a time and optionally where the output of each variable voltage reference is controlled by a microprocessor;
wherein at least one active component may be placed in a reduced power state, wherein when in the reduced power state it consumes less than or equal to 50% of the power in a full power state and
wherein the detector and amplifier circuit has a bandwidth of at least 200 kHz.

11. The pulse oximeter of claim 10, wherein the plurality of variable voltage references correspond to variable voltage references employed for red light, infrared light, and dark.

12. The pulse oximeter of claim 9, wherein the microprocessor controls the switching circuit controlling the detector and amplifier circuit and is optionally configured to signal the switching circuit to interrupt the flow of power between sampling bursts.

13. The pulse oximeter of claim 9, wherein the microprocessor controls the multiplexing switch of the voltage reference generator.

14. The pulse oximeter of claim 9, wherein the data comprises blood oxygen saturation, heart rate, and/or respiration rate, optionally collated with metadata such as time and date data.

15. The pulse oximeter of claim 9, wherein the microprocessor performs operations during at least some of the time the detector circuit is off.

16. The pulse oximeter of claim 9, wherein the pulse oximeter has a display for displaying data.

17. The pulse oximeter of claim 9, wherein the data can be transmitted to a remote network, smartphone, or other computer through a physical connection or a wireless connection.
